(19) United States
(12) Patent Application Publication
(10) Pub. No.: US 2006/0160895 A1
(43) Pub. Date: Jul. 20, 2006

(54) Anti-cancer agents

(76) Inventors: Weihui Huang, Shanghai (CN); Yumin Cui, Shanghai (CN); Jian Zuo, Shanghai (CN); Weihan Zhang, Shanghai (CN)

Correspondence Address: FISH & RICHARDSON PC, P.O. BOX 1022, MINNEAPOLIS, MN, 55440-1022, US

(21) Appl. No.: 11/292,321

(22) Filed: Dec. 1, 2005

Related U.S. Application Data
(60) Provisional application No. 60/632,615, filed on Dec. 2, 2004.

(51) Int. Cl. A61K 31/22; A61K 31/12

(52) U.S. Cl. 514/548; 514/690

(57) ABSTRACT

This invention relates to a method of treating gastric adenocarcinoma or lung carcinoma by administering to a subject in need thereof an effective amount of an effective amount of a diterpene compound of the following formula: 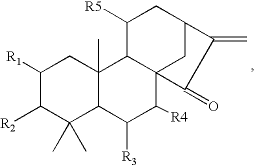
wherein R₁, R₂, R₃, R₄, and R₅ are defined herein.

### CROSS REFERENCE TO RELATED APPLICATIONS

Pursuant to 35 USC § 119(e), this application claims priority to U.S. Provisional Application Ser. No. 60/632,615, filed Dec. 2, 2004, the contents of which are incorporated herein by reference.

### BACKGROUND

Cancer, a leading fatal disease, features an abnormal mass of malignant tissue resulting from excessive cell division. More specifically, cancer cells proliferate in defiance of normal restraints on cell growth, and invade and colonize territories normally reserved for other cells.

Modes of cancer therapy include chemotherapy, surgery, radiation, and combinations of two or more of these treatments. Chemotherapy typically involves use of a compound that inhibits cancer cell growth. While many chemotherapeutic agents have been developed, there remains a need for more effective chemotherapy.

### SUMMARY

This invention is based on a surprising discovery that Lushanrubescensin A (LuA), Lushanrubescensin B (LuB), and Lushanrubescensin E (LuE) effectively inhibit the growth of gastric adenocarcinoma or lung carcinoma cells.

Thus, this invention relates to a method of treating cancer, such as gastric adenocarcinoma or lung carcinoma. The method is conducted by administering to a subject in need thereof an effective amount of a diterpene compound of Formula I: 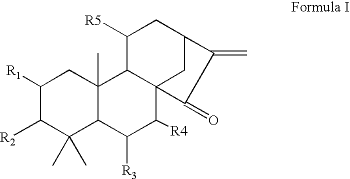
in which each of R₁, R₂, R₃, R₄ and R₅ is, independently, H, OH, OC(O)R, or OR, R being C₁₋₅ alkyl.

Referring to the Formula I, one subset of the diterpene compounds feature that each of R₁, R₂, and R₃, independently, is OC(O)CH₃; and R₄ is H or OH. Another subset of the diterpene compounds feature a stereospecificity shown in Formula II below:

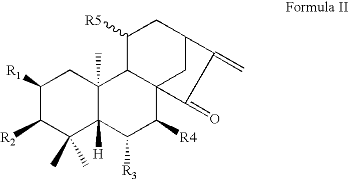

In the Formula II, each of R₁, R₂, and R₃, independently, can be OC(O)CH₃; and R₄can be H or OH.

Set forth below are three exemplary diterpene compounds that can be used to practice this method:

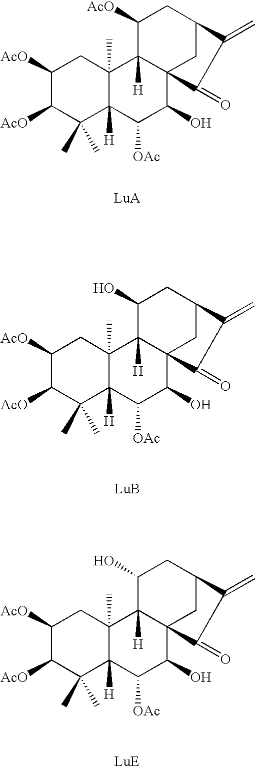

Also within the scope of this invention is a composition containing a diterpene compound of the Formula I and a pharmaceutically acceptable carrier for treating cancer (such as gastric adenocarcinoma or lung carcinoma), as well as use of a diterpene compound of the Formula I for the manufacture of a medicament for treating cancer.

The term “C₁₋₅ alkyl” refers to a straight or branched hydrocarbon, containing 1-5 carbon atoms. Examples of C₁₋₅ alkyl groups include, but are not limited to, methyl, ethyl, n-propyl, i-propyl, n-butyl, i-butyl, and t-butyl. The alkyl may optionally substituted. Examples of a substituent include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkenyl, alkynyl, alkyloxy, aryl, heteroaryl, cyclyl, and heterocyclyl, in which the alkenyl, alkynyl, alkyloxy, aryl, heteroaryl cyclyl, and heterocyclyl may be further substituted.

Details of several embodiments of the invention are set forth in the description below. Other features, objects, and advantages of the invention will be apparent from the description, and also from the claims.

### DETAILED DESCRIPTION

This invention relates to a method of treating gastric adenocarcinoma or lung carcinoma by administering to a subject in need thereof an effective amount of one or more of the diterpene compounds of the Formula I. The term “an effective amount” refers to the amount that is required to confer the intended therapeutic effect in a subject. Effective amounts may vary, as recognized by those skilled in the art, depending on route of administration, excipient usage, and possible co-usage with other active agents. The term “treating” refers to administering a diterpene compound to a subject that has gastric adenocarcinoma or lung carcinoma, or has a symptom of such a cancer, or has a predisposition toward such a cancer, with the purpose to cure, heal, alleviate, relieve, alter, remedy, ameliorate, improve, or affect the cancer, the symptoms of the cancer, or the predisposition toward the cancer.

Some of the diterpene compounds used to practice this method are naturally occurring and can be isolated from plants. For example, LuA, LuB and LuE can be isolated from the leaves of Rabdosia rubescens Hara f. lushanensis Gao et Li. Others can be synthesized by methods well known in the art or prepared from the naturally-occurring compounds via simple transformations. The chemicals used in the isolation and synthesis of the diterpene compounds may include, for example, solvents, reagents, catalysts, and protecting group and deprotecting group reagents. The isolation and synthesis may also include steps to add or remove suitable protecting groups in order to ultimately obtain desired diterpene compounds. Synthetic chemistry transformations and protecting group methodologies (protection and deprotection) useful in synthesizing applicable diterpene compounds are known in the art and include, for example, those described in R. Larock, Comprehensive Organic Transformations, VCH Publishers (1989); T. W. Greene and P. G. M. Wuts, Protective Groups in Organic Synthesis, 3d Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Diterpene compounds used to practice the method of this invention may contain one or more asymmetric centers. Thus, they can occur as racemic mixtures, single enantiomers, individual diastereomers, diastereomeric mixtures. All such isomeric forms are contemplated.

To practice the method of this invention, one administers to a subject having gastric adenocarcinoma or lung carcinoma an effective amount of the above-described compound and a pharmaceutically acceptable carrier orally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

An oral composition can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers may be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% almond and about 70% white soft paraffin by weight.

A carrier in a pharmaceutical composition must be “acceptable” in the sense that it is compatible with active ingredients of the formulation (and preferably, capable of stabilizing it) and not deleterious to the subject to be treated. For example, solubilizing agents, such as cyclodextrins (which form specific, more soluble complexes with one or more of active compounds of the extract), can be utilized as pharmaceutical excipients for delivery of the active ingredients. Examples of other carriers include colloidal silicon dioxide, magnesium stearate, cellulose, sodium lauryl sulfate, and D&C Yellow # 10.

Suitable in vitro assays can be used to preliminarily evaluate the efficacy of one or more of the above-described diterpene compounds in inhibiting growth of gastric adenocarcinoma or lung carcinoma cells, e.g., gastric adenocarcinoma (AGS, BGC-823), lung carcinoma (A549), or lung adenocarcinoma (SPC-A-1). The compounds can further be examined for its efficacy in treating gastric adenocarcinoma or lung carcinoma by in vivo assays. For example, the compounds can be administered to an animal (e.g., a mouse model) having gastric adenocarcinoma or lung carcinoma and its therapeutic effects are then assessed. Based on the results, an appropriate dosage range and administration route can also be determined.

Without further elaboration, it is believed that the above description has adequately enabled the present invention. The following specific examples are, therefore, to be construed as merely illustrative, and not limitative of the remainder of the disclosure in any way whatsoever. All of the publications, including patents, cited herein are hereby incorporated by reference in their entirety.

Isolation and Identification of LuA, LuB, and LuE

Aerial herb of Rabdosia rubescens Hara f. lushanensis Gao et Li (200 g, debris) was immersed in 2000 ml of 95% ethanol at room temperature for three days and then filtered out. The herb debris was treated in the same manner twice. The ethanol was removed from the combined filtrates under vacuum, and the residue thus obtained was diluted in 200 ml of water and washed with 200 ml of petroleum ether. The petroleum ether layer was then removed and the aqueous layer was washed with 200 ml of ethyl acetate. The ethyl acetate layer was collected and dried to afford a crude extract. The crude extract was loaded onto a silica gel column (8 cm×3 cm) and eluted with a gradient solution system of petroleum ether/acetone (9:1, 500 ml→8:2, 500 ml→7:3, 500 ml). Three fractions of eluate (500 ml each) were collected, containing 9:1, 8:2, and 7:3 petreolum ether/acetone, respectively. Strong UV absorbance at 254 nm was observed in each fraction. The first fraction, i.e., an eluate containing 9:1 petroleum ether/acetone, was concentrated and further purified using RP-18 chromatography with 50:50 methanol/water to afford pure LuA (37 mg, 0.02%). Similarly, the second fraction, i.e., an eluate containing 8:2 petreolum ether/acetone, and the third fraction, i.e., an eluate containing 7:3 petroleum ether/acetone, were concentrated and purified using RP-18 chromatography with 45:55 methanol/water and 40:60 methanol/water to afford pure LuB (59 mg, 0.03%) and LuE (81 mg, 0.04%), respectively.

Analytical data for LuA: m.p.=188˜190° C.; [α]_{D}¹³=−62.1° (c=0.99, pyridine); UV (EtOH) λₘₐₓ: 239 nm; IR (KBr): 3440, 1730, 1703, 1645, 1248, 1225 cm⁻¹; ¹H-NMR (C₅D₅N, 400 MHz) δ: 5.56 (1H, ddd, J=12.5, 4.0, 2.6 Hz), 5.30 (1H, d, J=2.6 Hz), 2.64 (1H, br), 5.54 (1H, dd, J=3.4, 1.7 Hz), 4.02 (1H, d, J=3.4 Hz), 2.40 (1H, br), 5.36 (1H, d, J=4.3 Hz), 3.00 (1H, br), 2.55 (1H, d, J=12.5 Hz), 1.48 (1H, dd, J=12.5, 3.6 Hz), 6.05 (1H, br), 5.35 (1H, br), 1.04 (3H, s), 1.12 (3H, s), 1.49 (3H, s); ¹³C-NMR (C₅D₅N, 100 MHz) δ: 212.7 (s), 170.6 (s), 170.4 (s), 169.8 (s), 169.0 (s), 150.2 (s), 114.6 (t), 77.6 (d), 73.1 (d), 71.0 (d), 68.3 (d), 67.6 (d), 55.0 (d), 50.0 (s), 42.0 (d), 40.8 (t), 39.8 (s), 38.3 (s), 38.3 (t), 37.3 (d) 34.5 (t), 28.0 (q), 23.2 (q), 21.2 (q), 20.9 (q), 20.6 (q), 20.6 (q), 20.6 (q); EIMS m/z: 535 [M+1]⁺, 474, 414, 354, 312, 294, 279.

Analytical data for LuB: m.p.=219˜221° C.; [α]_{D}¹⁹=−90° (c=0.1, methanol); IR (KBr): 3440, 1740, 1710, 1646, 1265˜1220 cm⁻¹; ¹H-NMR (C₅D₅N, 400 MHz) δ: 5.54 (1H, ddd, J=12.0, 4.2, 2.8 Hz), 5.20 (1H, d, J=2.8 Hz), 2.58 (1H, br), 5.45 (1H, dd, J=3.2, 1.4 Hz), 4.00 (1H, d, J=3.2 Hz), 2.55 (1H, br), 4.34 (1H, d, J=4.3 Hz), 3.03 (1H, br), 2.58 (1H, d, J=12.6 Hz), 1.46 (1H, dd, J=12.6, 3.4 Hz), 6.02 (1H, br), 5.36 (1H, br), 1.00 (3H, s), 1.13 (3H, s), 1.51 (3H, s); ¹³C-NMR (C₅D₅N, 100 MHz) δ: 213.6 (s), 170.6 (s), 170.6 (s), 169.9 (s), 151.0 (s), 112.9 (t), 77.8 (d), 73.7 (d), 71.5 (d), 67.9 (d), 65.0 (d), 59.1 (d), 49.9 (s), 42.1 (d), 40.8 (t), 40.8 (t), 39.4 (s), 38.3 (s), 38.1 (d), 35.2 (t), 28.0 (q), 23.3 (q), 21.3 (q), 21.3 (q), 21.0 (q), 20.7 (q); EIMS m/z: 492 [M]⁺, 432, 372, 312, 297, 279, 252, 240.

Analytical data for LuE: m.p.=215˜217° C.; [α]_{D}¹⁹=−77.5° (c=0.1, methanol); UV (EtOH) λmax: 238 nm; IR (KBr): 3550˜3370, 1740˜1720, 1710, 1650, 1265˜1225 cm⁻¹; ¹H-NMR (C₅D₅N, 400 MHz) δ: 5.74 (1H, d, J=3.0 Hz), 5.35 (1H, m), 4.68˜4.46 (1H, m), 3.08 (1H, br), 6.03 (1H, br), 5.29 (1H, br), 1.13 (3H, s), 1.10 (3H, s), 1.55 (3H, s); ¹³C-NMR (C₅D₅N, 100 MHz) δ: 208.0 (s), 170.8 (s), 169.9 (s), 150.8 (s), 111.4 (t), 81.4 (d), 69.0 (d), 65.0 (d), 63.8 (d), 63.6 (d), 49.2 (d), 48.8 (s), 44.5 (t), 41.3 (t), 39.7 (s), 38.5 (s), 38.5 (d), 37.9 (d) 37.9 (t), 28.2 (q), 22.9 (q), 21.5 (q), 20.9 (q), 20.2 (q); EIMS m/z: 434 [M]⁺, 416, 392, 374, 356, 314, 299, 296, 281, 253, 235.

Biological Assay

An in vitro assay was conducted to evaluate the efficacy of LuA, LuB, and LuE in inhibiting proliferation of cancer cells.

Four human tumor cell lines, i.e., gastric adenocarcinoma (AGS, BGC-823), lung carcinoma (A549), and lung adenocarcinoma (SPC-A-1), were purchased from the Cell Bank of Shanghai Institute of Cell Biology, Chinese Academy of Sciences, and cultured in Iscove's Modified Dulbecco's Medium (IMDM) containing 10% fetal bovine serum (FBS) in an incubator at 37° C. under 5% CO₂. Cells of 70˜80% confluence were trypsinized, resuspended in IMDM medium containing 10% FBS at 1×10⁵ cells/ml, and seeded in 96-well plates (100 μl in each well). The plates were incubated at 37° C. under 5% CO₂ overnight.

A DMSO solution containing LuA, LuB, or LuE was added to each “treatment” well. Efficacy of LuA, LuB, and LuE at six concentrations, 100, 30, 10, 3, 1, and 0.3 μg/ml, was studied. Wells, to each of which a DMSO solution (containing no LuA, LuB, or LuE) was added, were used as “control;” and wells, to each of which no LuA, LuB, LuE, or DMSO was added, were used as “background.” The plates were then incubated at 37° C. under 5% CO₂ for 48 hrs.

10 μl of 3-(4,5-dimethyl-thiazol-2-yl)-2,5-diphenyl tetrazolium bromide in phosphate buffered saline (PBS), 5 mg/ml, was added to all wells except the background wells. After incubation for 3-4 hrs, the cells were collected by spinning at 1000 rpm for 15 minutes, followed by removal of the supernatants by vacuum. The cells thus collected in each well, after wash with 150 μl of PBS, were placed in 150 μl of DMSO.

The plates were then placed on a shaker at 150 rpm for 15 minutes to dissolve the precipitate in each well. Absorbance was measured at 492 nm using a microplate reader.

Inhibition ratios for cells treated with LuA, LuB, and LuE, respectively, were calculated according to the following equation:

$$Inhibition ratio = 1 - \frac{Absorbance ( Treatment ) - Absorbance ( Background )}{Absorbance ( Control ) - Absorbance ( Background )} ⨯ 100 \%$$

A software program, XLfit (ID Business Solutions), was used to calculate the concentrations of LuA, LuB, and LuE required to reach 10, 20, . . . 90% inhibition (i.e., IC₁₀, IC₂₀, . . . IC₉₀) for each tested tumor cell line. The results show that LuA, LuB, and LuE effectively inhibited cancer cell lines AGS, BGC-823, A549, or SPC-A-1. Especially, both LuA and LuB exhibited particularly low IC₅₀ values for AGS, BGC-823, and A549.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for treating cancer, comprising administering to a subject in need thereof an effective amount of a compound of the following formula: 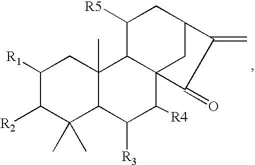
wherein each of R₁, R₂, R₃, R₄, and R₅, independently, is H, OH, OC(O)R, or OR, R being C₁₋₅ alkyl; and the cancer is gastric adenocarcinoma or lung carcinoma.

2. The method of claim 1, wherein each of R₁, R₂, and R₃, independently, is OC(O)CH₃.

3. The method of claim 2, wherein the cancer is gastric adenocarcinoma.

4. The method of claim 2, wherein the cancer is lung carcinoma.

5. The method of claim 1, wherein each of R₁, R₂, and R₃, independently, is OC(O)CH₃; and R₄ is OH.

6. The method of claim 5, wherein the cancer is gastric adenocarcinoma.

7. The method of claim 5, wherein the cancer is lung carcinoma.

8. The method of claim 1, wherein each of R₁, R₂ and R₃, independently, is OC(O)CH₃; and R₄ is H.

9. The method of claim 8, wherein the cancer is gastric adenocarcinoma.

10. The method of claim 8, wherein the cancer is lung carcinoma.

11. The method of claim 1, wherein the compound is of the following formula:

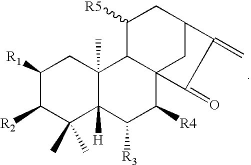

12. The method of claim 11, wherein each of R₁, R₂, and R₃, independently, is OC(O)CH₃.

13. The method of claim 12, wherein the cancer is gastric adenocarcinoma.

14. The method of claim 12, wherein the cancer is lung carcinoma.

15. The method of claim 11, wherein each of R₁, R₂, and R₃, independently, is OC(O)CH₃; and R₄ is OH.

16. The method of claim 15, wherein the cancer is gastric adenocarcinoma.

17. The method of claim 15, wherein the cancer is lung carcinoma.

18. The method of claim 11, wherein each of R₁, R₂, and R₃, independently, is OC(O)CH₃; and R₄ is H.

19. The method of claim 18, wherein the cancer is gastric adenocarcinoma.

20. The method of claim 18, wherein the cancer is lung carcinoma.

21. The method of claim 1, wherein the cancer is gastric adenocarcinoma.

22. The method of claim 21, wherein the compound is

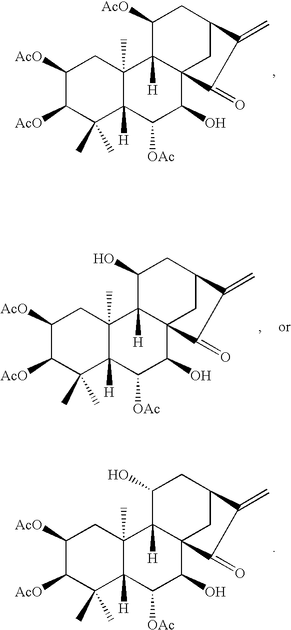

23. The method of claim 1, wherein the cancer is lung carcinoma.

24. The method of claim 23, wherein the compound is [chemical structure]
